(19) United States
(12) Patent Application Publication
(10) Pub. No.: US 2024/0148409 A1
(43) Pub. Date: May 9, 2024

(54) SYSTEMS, DEVICES, AND METHODS FOR CREATING CURVILINEAR TUNNELS IN BONE

(71) Applicant: AESCLEPIUS CORPORATION, La Jolla, CA (US)

(72) Inventors: Adam H. Hsieh, La Jolla, CA (US); Joe Ty Lin, San Diego, CA (US); Christopher Michael Rodriguez, Carlsbad, CA (US); Jacob Lev Rudelson, Del Mar, CA (US)

(21) Appl. No.: 18/207,746

(22) Filed: Jun. 9, 2023

Related U.S. Application Data
(63) Continuation of application No. PCT/US21/62776, filed on Dec. 10, 2021.
(60) Provisional application No. 63/124,313, filed on Dec. 11, 2020.

(51) Int. Cl. A61B 17/34; A61B 17/16

(52) U.S. Cl. CPC A61B 17/3472; A61B 17/1642; A61B 17/3494

(57) ABSTRACT

Systems, devices, and methods are provided for creating bone tunnels. In particular, described herein are embodiments of bone tunneling devices configured to create curvilinear tunnels in bone, as well as methods and devices relating thereto.

### CROSS-REFERENCE TO RELATED APPLICATIONS

This application is a continuation of International Patent Application No. PCT/US2021/062776, filed Dec. 10, 2021, which claims priority to and the benefit of U.S. Provisional Patent Application No. 62/124,313 filed Dec. 11, 2020, both of which are incorporated by reference herein in their entireties for all purposes.

### FIELD

The subject matter described herein relates generally to systems, devices, and methods for creating bone tunnels. In particular, described herein are embodiments of bone tunneling devices configured to create curvilinear tunnels in bone, as well as methods and devices relating thereto.

### BACKGROUND

Joint arthropathies (diseases that compromise joint function) are part of a steadily growing worldwide trend in chronic musculoskeletal disorders. In 2012, the Bone and Joint Initiative published findings that one out of every two Americans were diagnosed with musculoskeletal conditions, accounting for hundreds of billions of dollars in costs, which continue to grow annually. In 2018, the World Health Organization (WHO) identified the second largest contributor to global disability as musculoskeletal conditions. The increasing number of afflicted people and a continued rise in treatment costs point to a critical need for new technologies that provide more effective solutions to manage musculoskeletal ailments.

Joint arthropathies caused by soft tissue damage (e.g., tendon, ligament, and/or fibrocartilage tears) make up the majority of cases within the broader category of musculoskeletal conditions. Shoulder pain stands among the most common musculoskeletal complaints worldwide, with rotator cuff tears being the leading cause of shoulder disability. Other types of ligaments, tendon, and fibrocartilage injuries, such as labral tears, meniscus root tears, Achilles tendon avulsions, anterior cruciate ligament (ACL) ruptures, and lateral ankle ligament tears, among others, are somewhat less prevalent, but no less debilitating. Most of these injuries, whether due to tear size or lack of responsiveness to conservative treatment (e.g., physical therapy), require primary surgical repair. In 2014, the United States Agency for Healthcare Research and Quality (AHRQ) reported over 1.8 million invasive, therapeutic surgeries involving “muscle, tendon, soft tissue operating room procedures” and “incision or fusion of joint, or destruction of joint lesion” in the United States, which equates to 8.3% of the roughly 21.7 million total ambulatory and inpatient surgical procedures.

The goal of such repairs is to re-establish the position and direction of force transmission in these tissues in order to restore stability and motion to their respective joints. For soft tissue injuries, this can be achieved by re-attaching the torn areas of soft tissue (e.g., tendon, ligament, and/or fibrocartilage)—which naturally pull away from their anatomic insertion site upon injury—using a fixation method to create a stable connection and close contact between tissue and bone so that the interface can heal over time.

In some soft tissue surgical repair techniques, a bone tunnel is required either for the insertion of an implant, suture, or tissue. For instance, ACL reconstructions often employ the use of straight bone tunnels for both femoral and tibial fixation of graft tissue using interference screws and/or bone plugs. As another example, rotator cuff repairs may utilize a transosseous approach involving the creation of curvilinear or piece-wise linear bone tunnels through which sutures are passed to pull the torn tendon back to the bone.

Although these types of bone tunnels may be adequate in many cases, there may be situations, or newly developed technologies and approaches, that will require curvilinear bone tunnels that possess geometric features that are different from those that can be generated using currently available devices. Thus, needs exist for easily scalable systems, devices and methods that can achieve these objectives without the need for additional special equipment.

### SUMMARY

Provided herein are example embodiments of systems, devices and methods for generating curvilinear bone tunnels. According to some embodiments, a device for creating curvilinear tunnels in a bone is provided, wherein the device can comprise a housing having a distal end configured to interface with a surface of the bone; an impactor at least partially disposed within the housing, wherein the impactor is configured to be inserted into the bone and to create a curvilinear tunnel, and wherein the impactor comprises a rigid material and a curved geometry; an inner channel disposed within the housing and configured to guide the impactor into the bone; and an actuator comprising a propulsion mechanism configured to move the impactor.

According to other embodiments, a device for creating curvilinear tunnels in a bone is provided, wherein the device can comprise a housing having a distal end configured to interface with a surface of the bone; one or more curved needles at least partially disposed within the housing, wherein the one or more curved needles are configured to be inserted into the bone and to create a curvilinear tunnel, and wherein the one or more curved needles comprise a superelastic material; one or more straight hollow punches configured to introduce the one or more curved needles at a predetermined depth under the surface of the bone; one or more inner channels disposed within the housing, wherein each of the one or more inner channels is configured to guide a corresponding curved needle into the bone; a first set of one or more drivers configured to move the one or more straight hollow punches; and a second set of one or more drivers configured to move the one or more curved needles.

According to still other embodiments, a device for creating curvilinear tunnels in a bone is provided, wherein the device can comprise a housing having a distal end configured to interface with a surface of the bone; a flexible hollow shaft configured to bend during insertion into bone and to guide a path of a curvilinear tunnel; a flexible drill bit including an exposed head, wherein the exposed head is located at a tip portion of the flexible hollow shaft, and wherein the flexible drill bit includes a drill shaft configured to rotate within the flexible hollow shaft; a first set of one or more drivers to steer and extend the flexible hollow shaft; and a second set of one or more drivers to spin and extend the flexible drill bit.

The various configurations of these systems, methods and devices are described by way of the embodiments which are only examples. Other systems, devices, methods, features, improvements and advantages of the subject matter described herein are or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A is a perspective view of an example embodiment of a bone tunneling device with a curved impactor.

FIG. 1B is a side view of an example embodiment of a bone tunneling device with a curved impactor.

FIGS. 2A and 2B are side views of another example embodiment of a bone tunneling device with a curved needle in various configurations.

FIG. 2C is an exploded view of an example embodiment of a bone tunneling device with a curved needle.

FIGS. 3A and 3B are side views of another example embodiment of a bone tunneling device with two curved needles in various configurations.

FIGS. 4A and 4B are side views of another example embodiment of a bone tunneling device with a curved needle.

FIGS. 5A to 5F are side views of various example embodiments of curved needles.

FIG. 6 is a perspective view of an example embodiment of a bone tunneling device with a steerable shaft and a flexible drill bit.

FIG. 7 is an example block diagram for controlling the operation of an actuator in a bone tunneling device.

FIG. 8 is an example flow chart illustrating the method of use for the bone tunneling device in conjunction with or without bracing apparatuses for repairing the attachment of soft tissue to bone.

FIG. 9 is an example flow chart illustrating a method of manufacture for a curved needle.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for generating curvilinear tunnels in bone. Accordingly, a tunneling device is provided for creating a curved bone tunnel. In certain embodiments, the tunneling device can include a curved channel or guide tube configured to guide a pointed impactor along a predetermined path. In some embodiments, for example, one or more ends of a curved track or guide tube abut a target area of a bone surface, wherein the target area comprises one or more predetermined entry and exit points of a tunnel to be created in the bone. In some embodiments, a tunneling device can also include a means for propelling the impactor, for example, through the use of a pneumatic, magnetic, electrical, or mechanical mechanism, or a combination thereof, whether automated or manually operated.

In other embodiments, a tunneling device includes a sharp tipped needle that can be made of a superelastic material. One example of a superelastic material is nickel-titanium alloy, also known as nitinol. According to the embodiments, the sharp tipped needle can be retracted into a curved or straight guide tube, and then extended from the guide tube either by a manual or automated mechanism within the tunneling device to enable the sharp tipped needle to extend out of the guide tube, thereby assuming its curved configuration, to create a curvilinear bone tunnel.

In another embodiment, a tunneling device includes a steerable component and an extending component that enables the device to advance into the bone along a controllable path.

For each and every embodiment of a method disclosed herein, systems and devices capable of performing each of those embodiments are covered within the scope of the present disclosure. For example, embodiments of tunneling devices for creating a bone tunnel are disclosed, and these devices can each have one or more internal propulsion mechanisms.

### Example Embodiments of Tunneling Devices and Methods Relating Thereto

Example embodiments of tunneling devices for creating a bone tunnel, and methods relating thereto, will now be described.

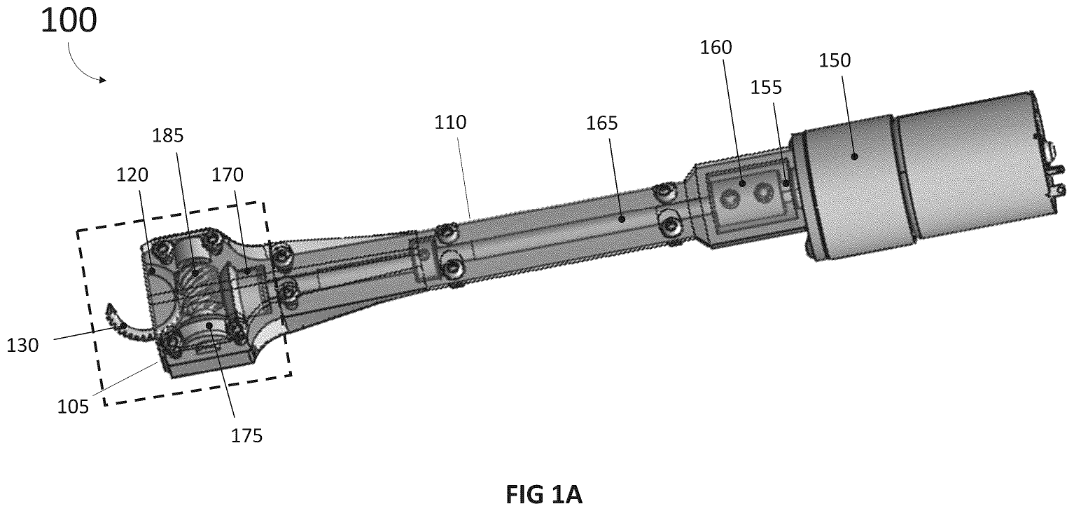

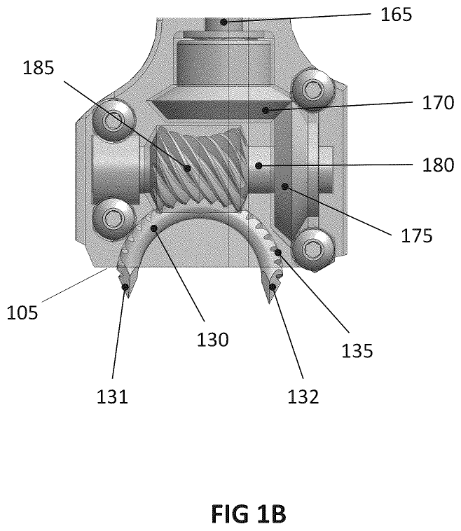

FIG. 1A is a perspective view depicting an example embodiment of a tunneling device 100 for creating one or more curvilinear tunnels in a bone material. According to some embodiments, tunneling device 100 includes a housing 110 comprising a distal end having at least one surface 105 configured to interface with a bone material, a channel 120 disposed within housing 110, and a curved impactor 130 configured to travel within channel 120. Channel 120 is shown to be circular in cross-section, but those of skill in the art will appreciate that the cross-sectional shape of channel 120 and impactor 130, configured to travel therein, can be of any geometry. According to some embodiments, the path of channel 120 within tunneling device 100 can have an arcuate geometry. Furthermore, as seen in FIG. 1A, tunneling device 100, including housing 110 and channel 120 disposed therein, comprises a semi-circular shape subtending an angle of 180 degrees. Those of skill in the art, however, will appreciate that tunneling device 100, housing 110, or channel 120, can comprise a circular arc subtending an angle of more or less than 180 degrees, or can have a non-circular shape. In addition, the length of curved impactor 130 can be either greater than or less than the length of channel 120 of tunneling device 100.

In some embodiments, an internal propulsion mechanism transfers energy to impactor 130 to generate bidirectional motion within channel 120. The internal propulsion mechanism can comprise technology used to generate motion including, but not limited to, an electromechanical actuator, a piezoelectric actuator, an electrical induction actuator, a magnetic propulsion actuator, a pneumatic propulsion actuator, a hydraulic propulsion actuator, a mechanical propulsion (e.g., linkages, gears, etc.) actuator, or any combination thereof. Furthermore, according to some embodiments, the internal propulsion mechanism can be fully automated, partially automated, or manually powered. FIG. 1A depicts a perspective partial cross-sectional view of an example embodiment of tunneling device 100 comprising a direct current (DC) motor 150 configured to move impactor 130. According to one aspect of the embodiments, DC motor 150 drives a rotating shaft 155. Shaft 155 inserts into a coupler 160, which is coupled with drive shaft 165, which is configured to transmit torque from DC motor 150 to miter gear 170.

FIG. 1B is a perspective view depicting a distal end of tunneling device 100. As best seen in FIG. 1B, miter gear 170 is operatively engaged with a second complementary miter gear 175. Rotation of second miter gear 175 turns shaft 180, as well as worm screw 185. In a manner analogous to a worm drive where a worm screw turns a worm gear, the direction of rotation of worm screw 185 drives the forward or backward direction of motion of impactor 130, which possesses teeth 135 complementary to the worm screw 185 along its outer surface. According to another aspect of the embodiments, impactor 130 includes one or more pointed ends 131 and 132, which are configured such that the energy upon striking or burrowing through a bone material can cause a bone tunnel to lengthen. Although the embodiment is depicted in FIG. 1B as using a throated worm screw 185 and a non-throated impactor 130, those of skill in the art will recognize that worm screw 185 and impactor 130 can each be throated or non-throated, and such embodiments are within the scope of the present disclosure.

According to some embodiments, impactor 130 can comprise a solid component including one or more solid conical tips 131 and 132 on each end with teeth 135 complementary to worm screw 185. Those of skill in the art, however, will appreciate that impactor 130 can possess one or more tips 131 and 132 having a different geometry including, but not limited to, pyramidal, hollow cylindrical, hemispherical, or truncated conical tip, along with other tip features, such as flutes and tapers. Additionally, the cross sectional geometry of the impactor 130 is depicted as circular, but can be of any shape including, but not limited to, elliptical, polygonal, or an irregular shape. The impactor 130 can also be hollow or partially hollow. Some embodiments of tunneling device 100 may not include teeth 135, but can introduce other physical features or properties of impactor 130 in order to achieve motion by said means of propulsion. Certain embodiments of tunneling device 100 can use an impactor 130 of appropriate size, geometry, and properties to form a curved bone tunnel, and leave the impactor within the tunnel as an implantable device after the tunnel is formed.

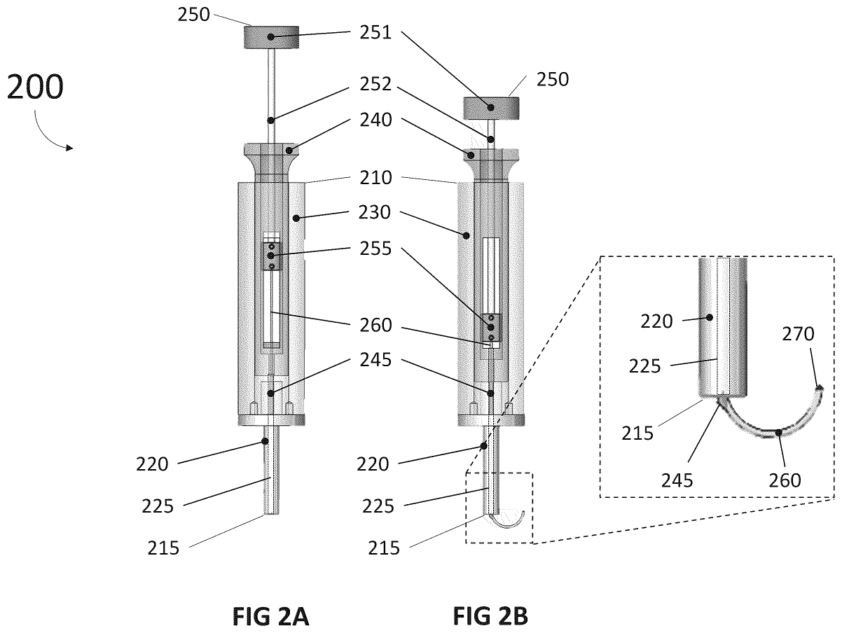

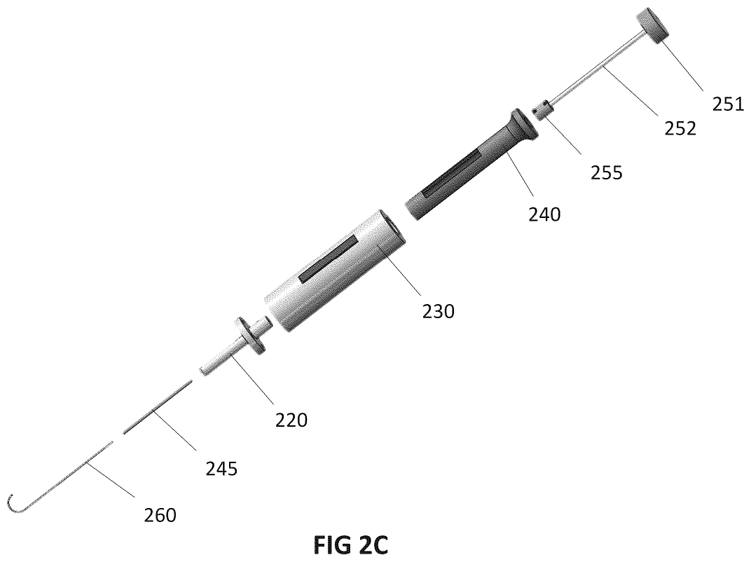

FIGS. 2A and 2B depict a side view of another embodiment of tunneling device 200 in retracted and fully deployed, extended configurations, respectively. FIG. 2C depicts an exploded view of tunneling device 200. According to one aspect of many embodiments, tunneling device 200 can comprise a curved needle 260 configured to be driven into bone to create a curved bone tunnel. In some embodiments, curved needle 260 can comprise a superelastic material, one of numerous examples of which include nickel-titanium alloy, also known as nitinol. Furthermore, according to some embodiments, tunneling device 200 can comprise a housing 210, which can include a distal end comprising at least one surface 215 configured to interface with a bone material, a shaft 220 containing a channel 225 through which the curved needle 260 is extended and retracted, and a main body 230.

According to an aspect of the embodiments, numerous subassemblies can be provided to control the depth and formation of the curved tunnel to be created. In some embodiments, for example, a first subassembly comprises first plunger 240 and hollow punch 245, both of which are mutually engaged. First plunger 240 is configured to slide freely through main body 230, and hollow punch 245, in turn, is configured to slide freely within channel 225 in shaft 220. In this regard, when a first set of drivers pushes first plunger 240, whether by a screw mechanism or by impact, and whether by automation or by manual operation, hollow punch 245 will extend from an orifice on surface 215 to enable hollow punch 245 to enter the bone material. The extension of hollow punch 245 from the orifice is best seen in the inset of FIG. 2B showing a closeup of the region of tunneling device 200 that interfaces with bone. The extended length of hollow punch 245 can be zero (no extension) or any finite length that can be accommodated by the housing 210 and by the needle 260 within it. Although hollow punch 245 is depicted as circular in cross section, those of knowledge in the art will recognize that the cross section of hollow punch 245 can be of any geometry, including, but not limited to, elliptical, polygonal, or any irregular shape.

In some embodiments, a second subassembly comprises a second plunger 250 attached to a curved needle 260 via a connector 255. Second plunger 250 further comprises a plunger head 251 and plunger shaft 252. When a second set of drivers pushes second plunger 250, whether by a screw mechanism or by impact, and whether by automation or by manual operation, through the first subassembly, this can cause curved needle 260 to extend through the hollow punch 245 in shaft 220 by way of connector 255. The tip 270 of curved needle 260 emerges from the end of hollow punch 245 at the surface of the bone if hollow punch has an extension of zero, or below the surface of bone if the hollow punch is extended a non-zero distance.

As described earlier, according to some embodiments, curved needle 260 can comprise a superelastic material that enables it to be straightened with little or no permanent deformation when retracted within the hollow punch 245 in shaft 220 of the housing 210. Although curved needle 260 is shown to be circular in cross section, those of knowledge in the art will appreciate that the cross section of curved needle 260 can be of any geometry, including, but not limited to, elliptical, polygonal, or any irregular shape. Also, this embodiment is depicted and described with connector 255, but those with skill in the art will appreciate that connector 255 can be omitted if curved needle 260 can be directly attached to plunger shaft 252. Those of skill in the art will also recognize that the tip 270 of curved needle 260 can be larger or smaller in diameter than the curved needle. Those of skill in the art will further recognize that the tip 270 of curved needle 260 can have any geometry including, but not limited to, conical, pyramidal, hollow cylindrical, hemispherical, or truncated conical tip, along with other tip features, such as flutes and tapers.

Those of knowledge in the art will appreciate that the extension of the hollow punch 245 in tunneling device 200 can occur after surface 215 has been placed in contact with bone. Alternatively, hollow punch 245 in tunneling device 200 can be extended before surface 215 comes into contact with bone, and then can be used to penetrate the surface of bone forcibly while already extended. Those of knowledge in the art will also appreciate that the depth of which the hollow punch 245 enters bone can be user-adjustable or preset, and can be zero or any depth that can be accommodated by the design of tunneling device 200.

Referring still to FIGS. 2A and 2B, housing 210 is illustrated to comprise separate shaft 220 and main body 230 components. Those of skill in the art, however, will appreciate that the housing 210 in tunneling device 200 can be manufactured with more or fewer components, or as a single component. Similarly, the first subassembly of tunneling device 200 can be of any size and shape with fewer or more components, with its primary purpose being to interface with and move hollow punch 245. Likewise, the second subassembly can be of any size and shape with fewer or more components, with its primary purpose being to interface with and move curved needle 260 in tunneling device 200. Additionally, those of skill in the art will appreciate that all channels—shown to be straight within the shaft—can be curved, can have any cross-sectional geometry, and can extend into the main body. Those of skill in the art will also appreciate that the curved needle 260 in tunneling device 200 can be of any length, curvature, and tortuosity, and can be of any cross-sectional shape and size. Those with skill in the art will recognize that mechanisms for extension and retraction of the needle, e.g., using a pneumatic, magnetic, electrical, or mechanical actuator, can be either external to or integral with the device, and moreover, can be automated or manually operated. Such a mechanism can be incorporated within or outside of the device, in conjunction with or in place of other components in each subassembly.

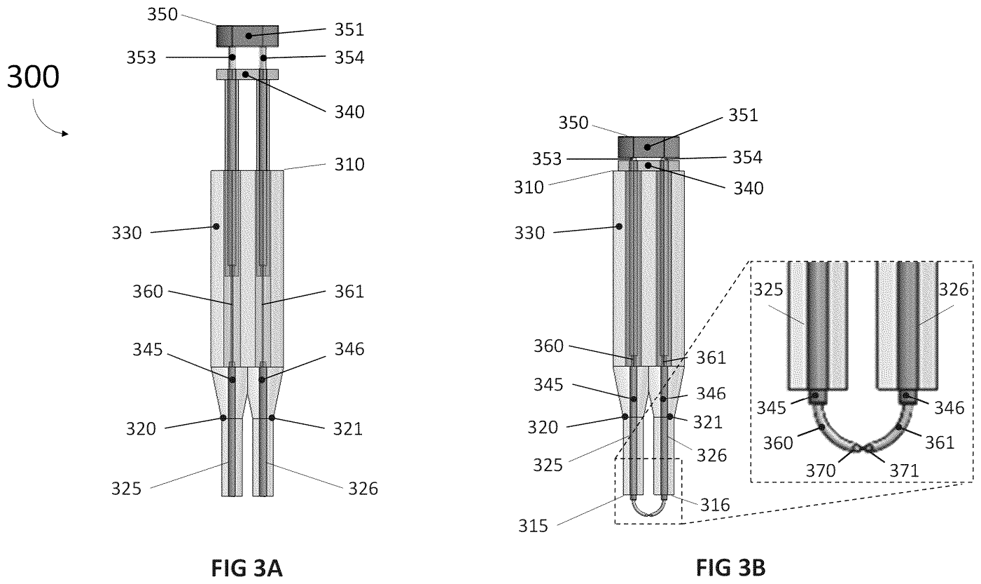

FIGS. 3A and 3B depict side views of another embodiment of tunneling device 300 in retracted and fully deployed configurations, respectively. Tunneling device 300 utilizes similar principles as tunneling device 200, but employs two curved needles 360 and 361 that are configured to meet when fully extended to form a continuous curvilinear bone tunnel. In some embodiments, curved needles 360 and 361 can comprise a superelastic material, one of numerous examples of which include nickel-titanium alloy, also known as nitinol. Furthermore, according to some embodiments, tunneling device 300 comprises a housing of unibody construction 310, in which the two shafts 320 and 321, together with main body 330, are regions within a single component. Main body 330 has two channels, one for each curved needle. Each channel continues from the main body 330 into one of the two shafts 320 and 321.

Numerous subassemblies can be provided to control the depth and formation of the curved tunnel to be created. In some embodiments, for example, a first subassembly comprises a first plunger 340 that includes two plunger shafts, each of which is directly engaged with one of the hollow punches 345 and 346. First plunger 340 is configured to slide freely through a corresponding channel within main body 330, and each of the hollow punches 345 and 346 are configured to slide freely within their respective channels 325 and 326 in corresponding shafts 320 and 321. In this regard, when a first set of drivers pushes first plunger 340, whether by a screw mechanism or by impact, and whether by automation or by manual operation, hollow punches 345 and 346 will each extend from an orifice on respective surfaces 315 and 316 to enable hollow punches 345 and 346 to enter the bone material. The extension of hollow punches 345 and 346 from their respective orifices is best seen in the inset of FIG. 3B showing a closeup of the region of tunneling device 300 that interfaces with bone. The extended length of hollow punches 345 and 346 can be zero (no extension) or any finite length that can be accommodated by the housing and by the needle within it. Although hollow punches 345 and 346 are depicted as circular in cross section, those of knowledge in the art will recognize that the cross section of hollow punches 345 and 346 can be of any geometry, including, but not limited to, elliptical, polygonal, or any irregular shape. Also, although hollow punches 345 and 346 are depicted as extending the same length, those of knowledge in the art will recognize that the two hollow punches can extend to two different lengths.

A second subassembly comprises a second plunger 350 attached to curved needles 360 and 361, respectively. Second plunger 350 further comprises plunger head 351 and plunger shafts 353 and 354. According to one aspect of the embodiments, when a second set of drivers pushes second plunger 350, whether by a screw mechanism or by impact, and whether by automation or by manual operation, through main body 330, this causes curved needles 360 and 361 to extend through hollow punches 345 and 346 in corresponding shafts 320 and 321. The tip 370 of curved needle 360 and tip 371 of curved needle 361 emerge from the ends of their respective hollow punches 345 and 346 at the surface of the bone if hollow punch has an extension of zero, or below the surface of bone if either or both hollow punches 345 and 346 are extended a non-zero distance.

As described earlier, according to some embodiments, curved needles 360 and 361 can comprise a superelastic material that enables the needles to be straightened with little or no permanent deformation when retracted within hollow punches 345 and 346 in shafts 320 and 321 of the housing 310. Although curved needles 360 and 361 are shown to be circular in cross section, those of knowledge in the art will appreciate that the cross sections of curved needles 360 and 361 can be of any geometry, including, but not limited to, elliptical, polygonal, or any irregular shape. Those of skill in the art will also recognize that the tips 370 and 371 of respective curved needles 360 and 361 can be larger or smaller in diameter than the curved needle. Those of skill in the art will further recognize that the tips 370 and 371 of respective curved needle 360 and 361 can have any geometry including, but not limited to, conical, pyramidal, hollow cylindrical, hemispherical, or truncated conical tip, along with other tip features, such as flutes and tapers.

Those of knowledge in the art will appreciate that the extension of the hollow punches 345 and 346 in tunneling device 300 can occur after surfaces 315 and 316 have been placed in contact with bone. Alternatively, hollow punches 345 and 346 in tunneling device 300 can be extended before surfaces 315 and 316 come into contact with bone, and then can be used to penetrate the surface of bone forcibly while already extended. Those of knowledge in the art will also appreciate that the depth of which the hollow punches 345 and 346 enter bone can be user-adjustable or preset, and can be zero or any depth that can be accommodated by the design of tunneling device 300.

Housing 310 is shown to comprise a single component with two shafts 320 and 321 and a main body 330. Those of skill in the art, however, will appreciate that the housing in tunneling device 300 can be manufactured with more components, or with a single shaft comprising multiple channels. Similarly, the first subassembly of either tunneling device 300 can be of any size and shape with fewer or more components, with its primary purpose being to interface with and move hollow punches 345 and 346. Likewise, the second subassembly can be of any size and shape with fewer or more components, with its primary purpose being to interface with and move curved needles 360 and 361 in tunneling device 300. Additionally, those of skill in the art will appreciate that all channels—shown to be straight within the shaft—can be curved, can have any cross-sectional geometry, and can extend into the main body. Those of skill in the art will also appreciate that the curved needles 360 and 361 in tunneling device 300 can be of any length, curvature, and tortuosity, and can be of any cross-sectional shape and size. Those with skill in the art will recognize that mechanisms for extension and retraction of the needle, e.g., using pneumatic, magnetic, electrical, or mechanical actuators, can be either external to or integral with the device and, moreover, can be automated or manually operated. Such a mechanism can be incorporated within or outside of the device, in conjunction with or in place of other components in each subassembly.

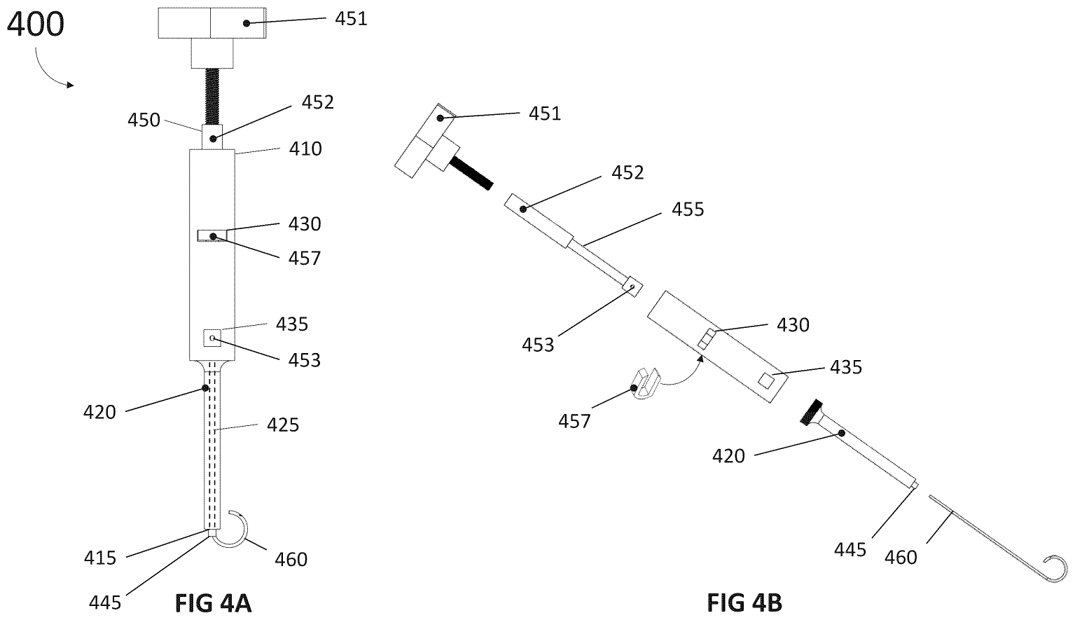

FIG. 4A depicts a side view of another embodiment of tunneling device 400 in a fully deployed configuration. FIG. 4B depicts an exploded view of tunneling device 400. Tunneling device 400 utilizes similar principles as tunneling device 200, but additionally comprises several parts for the purposes of greater functionality of the device. Similar to tunneling device 200, according to one aspect of many embodiments, tunneling device 400 can comprise a housing 410, which can include a distal end comprising at least one surface 415 configured to interface with a bone material and a hollow punch 445 of fixed length configured to penetrate bone material. According to some embodiments, hollow punch 445 can be a component coupled with shaft 420. Those of knowledge in the art can appreciate that the length of hollow punch 445 can be zero or any finite length that can be accommodated by the housing 410 and by the needle 460 within it. Although hollow punch 445 is depicted as circular in cross section, those of knowledge in the art will recognize that the cross section of hollow punch 445 can be of any geometry, including, but not limited to, elliptical, polygonal, or any irregular shape. A channel 425, which is continuous within the hollow punch 445 and the shaft 420, allows curved needle 460 to be extended and retracted. In some embodiments, curved needle 460 can comprise a superelastic material, one of numerous examples of which include nickel-titanium alloy, also known as nitinol.

Referring still to FIGS. 4A and 4B, according to another aspect of the embodiments, a subassembly comprises a plunger 450 directly coupled with a curved needle 460. In some embodiments, a first window 430 can be made in housing 410 to provide access to the attachment point 453 between plunger 450 and curved needle 460. Plunger 450 further comprises a T-shaped plunger head 451 that is coupled with plunger shaft 452. When a set of drivers pushes plunger 450, whether by a screw mechanism or by impact, and whether by automation or by manual operation, this can cause curved needle 460 to extend through the hollow punch 445 in shaft 420. The T-shaped plunger head 451 is T-shaped to assist in manual retraction of the plunger, in situations where manual retraction is used. Those of knowledge in the art, however, will appreciate that the plunger head can also have other geometries or mechanisms that can assist in manual retraction including, but not limited to, a ring-shaped puller to accommodate one or more fingers, or a trigger activated by squeezing of the hand. Referring to FIG. 4B, the plunger shaft 452 possesses a neck area 455 that is smaller in width relative to the plunger shaft. A U-shaped insert 457 with a gap between arms of the U that is larger than the width of the neck area 455 and smaller than the width of the plunger shaft 452 is placed into a second window 435 in housing 410 such that the arms of U-shaped insert 457 straddle either side of the neck area 455. This ensures that the travel distance of plunger 450 is constrained to the length of neck area 455, and that the plunger 450 cannot be removed while the U-shaped insert 457 is seated in the second window 435 of housing 410. Although the mechanism for limiting travel distance is depicted as a U-shaped insert 457 straddling either side of neck area 455 of plunger shaft 452, those of skill in the art will recognize that the travel distance can also be limited by other means, including, but not limited to, a pin, rod, or beam inserted through second window 435 of different shape in housing 410 and through a slot along the length of plunger shaft 452.

Each of the example embodiments of tunneling device 200, 300, and 400 comprises one or more curved needles. As described earlier, these curved needles 260, 360, 361, and 460 can comprise a superelastic material with cross-sectional geometries of any shape and size; with any length, curvature, and tortuosity; with respective tips 270, 370, 371, and 470 of any geometry and comprising other tip features. Curved needles 260, 360, 361, and 460 can further comprise features on the side(s) of the curved needle, along the length of the curved needle, and near or at the curved needle tip to enable additional functionality, including, but not limited to, the ability to capture objects extrinsic to the device.

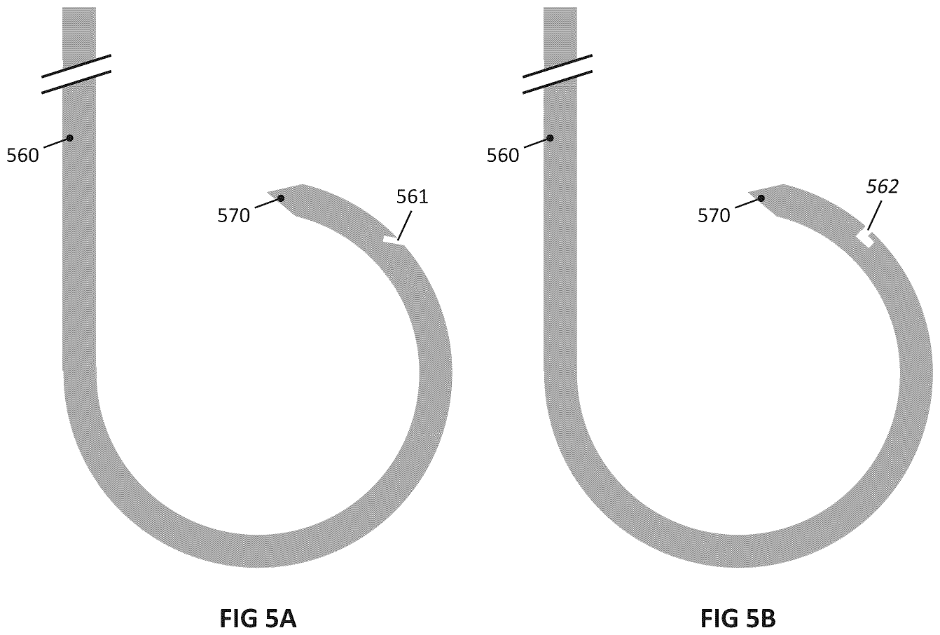

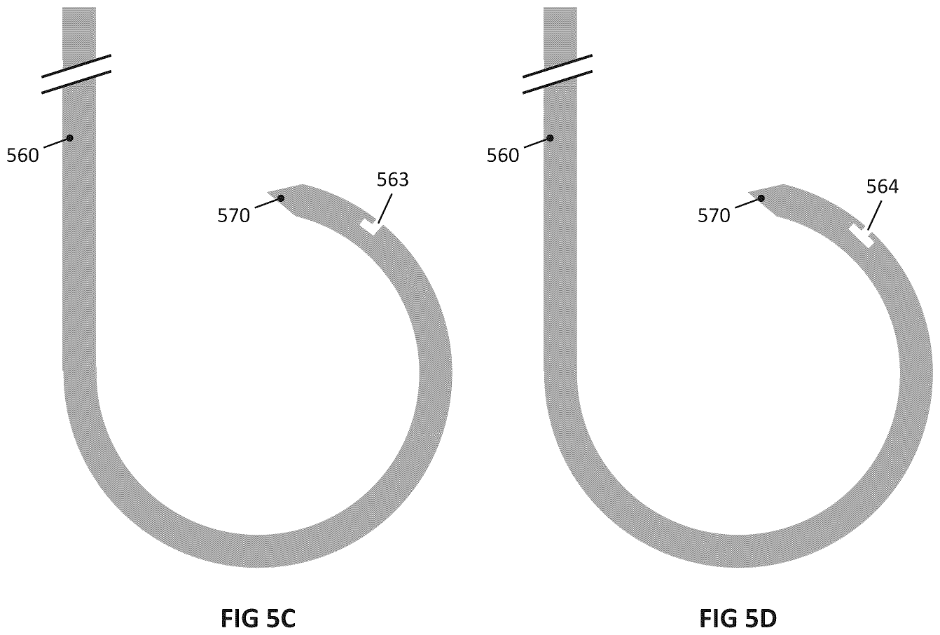

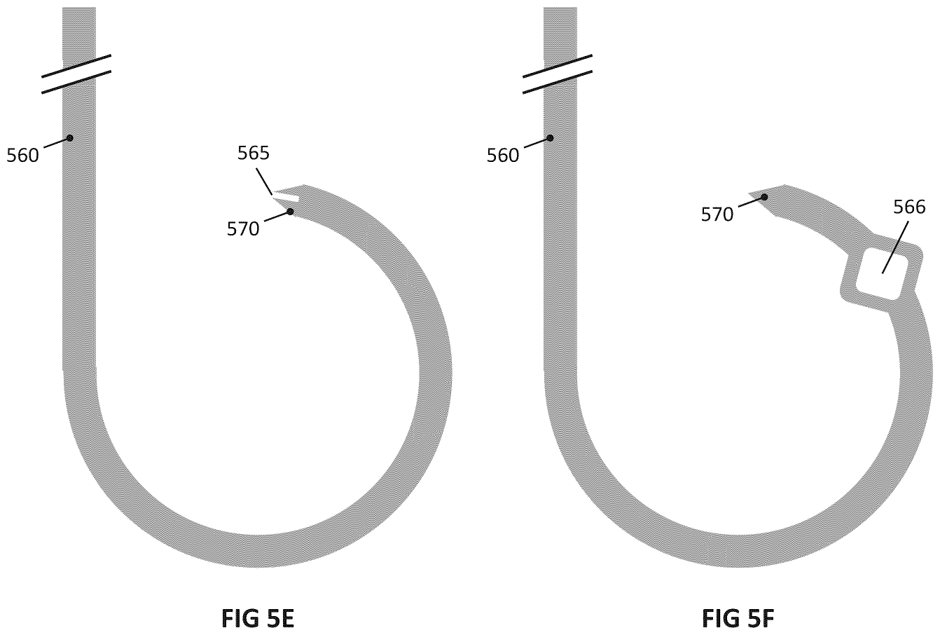

FIG. 5A depicts one example embodiment of a curved needle 560 comprising an angled slot feature 561, near the curved needle tip 570, forming a sharp hook-like feature that can capture a thin wire or suture. In many of the embodiments, angled slot feature 561 can be disposed on a convex surface of curved needle 560. In other embodiments, angled slot feature 561 can be disposed elsewhere on curved needle 560, such as, e.g., the concave surface of curved needle 560. Furthermore, in some embodiments, angled slot feature 561 and curved needle tip 570 form an arc along the length of curved needle 560, and this arc subtends an angle of approximately 45 degrees. Those of skill in the art will recognize, however, that the angled slot feature 561 can be positioned anywhere along curved needle 560 to form a longer or shorter arc with curved needle tip 570. Also, in this example embodiment, the centerline of the angled slot feature 561 is oriented 30° relative to the tangent of the curved needle at the centerline of the slot, pointed toward the needle tip, with a penetration depth of 33% of the curved needle diameter. Those of knowledge in the art will appreciate that the slot can instead be angled away from the tip, can be positioned anywhere along the length of curved needle 560, can be oriented at an angle of any value relative to the tangent of the curved needle, and can be of any depth.

FIGS. 5B, 5C, and 5D respectively depict additional example embodiments of features 562, 563, and 564 on the side or surface of curved needle 560 that are intended to impart other functionalities to the device. FIG. 5B depicts an example embodiment of curved needle 560 comprising a boot-shaped feature 562 with the “toe” part of the feature pointing away from the needle tip 570. FIG. 5C depicts an example embodiment of curved needle 560 comprising a boot-shaped feature 563 with the “toe” part of the feature pointing toward the needle tip 570. FIG. 5D depicts an example embodiment of curved needle 560 comprising a T- or mushroom-shaped feature 564.

FIG. 5E depicts an example embodiment of curved needle 560 that comprises a simple slotted fork feature 565 at the needle tip 570.

FIG. 5F depicts an example embodiment of curved needle 560 that comprises an orifice 566 that can be shaped like a diamond or a human eye. When the curved needle enters into a tight space, such as a tunnel or tube or channel, the bowed out segments of curved needle that form orifice 566 will pinch together to enable the capture of objects extrinsic to the device.

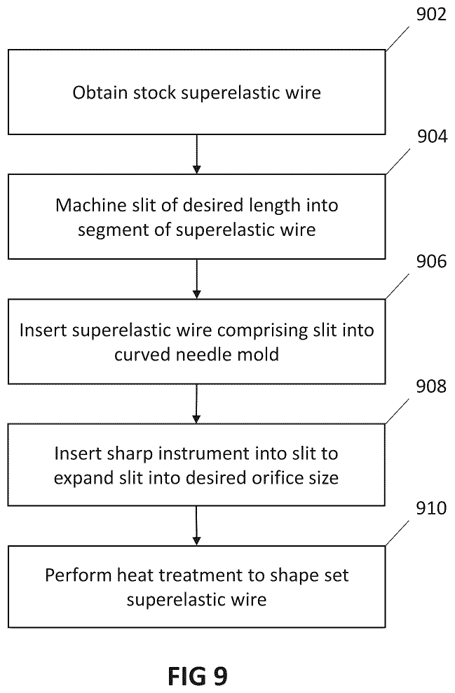

With respect to the manufacture of curved needles, such as those described in the present disclosure, FIG. 9 depicts a flow diagram of an example embodiment of a method for manufacture. When using a superelastic material for manufacturing curved needle 560, one example method for creating an orifice 566 is to obtain straight or coiled wire stock of the material (Step 902). From the superelastic wire stock, a segment of superelastic wire can then be machined by various techniques including, but not limited to, electrical discharge machining (EDM) or laser cutting, to introduce a slit of desired length parallel to the length of superelastic wire (Step 904). The superelastic wire can then be placed into a mold that will hold the intended shape of the curved needle 560 (Step 906). Once the wire stock is secured in the mold, the tip of a sharp instrument can be inserted into the slit to expand the slit to the desired width of orifice 566 (Step 908). With the sharp instrument still in place within the expanded slit of the superelastic wire, the mold comprising the superelastic wire and the sharp instrument can then be subjected to heat treatment, in order to shape set the superelastic wire (Step 910). Following heat treatment, after the sharp instrument is removed from the newly shape set orifice and the superelastic wire is removed from the mold, the resulting curved wire comprising the orifice can then be subjected to any additional processes to complete curved needle 560. Those of skill in the art will recognize that the precise order of steps prior to heat treatment can vary, and that interspersing multiple heat treatment steps between different physical manipulations of superelastic wire can be performed, to achieve the same results.

All of these features shown in FIGS. 5A to 5F are intended to illustrate general concepts of example embodiments and in no way should they serve to limit the types of features that can be comprised by the curved needle. Those with knowledge of the art will appreciate that features can be of any shape and size, can be located on any aspect of the curved needle cross-section, can be facing any direction and orientation, and can be at any position along the length of the curved needle, including any straight portions of the curved needle 560. Those with knowledge of the art will also appreciate that features depicted in FIGS. 5A to 5F can have parallel edges, non-parallel edges, curved or filleted or rounded edges, and wider or narrower openings.

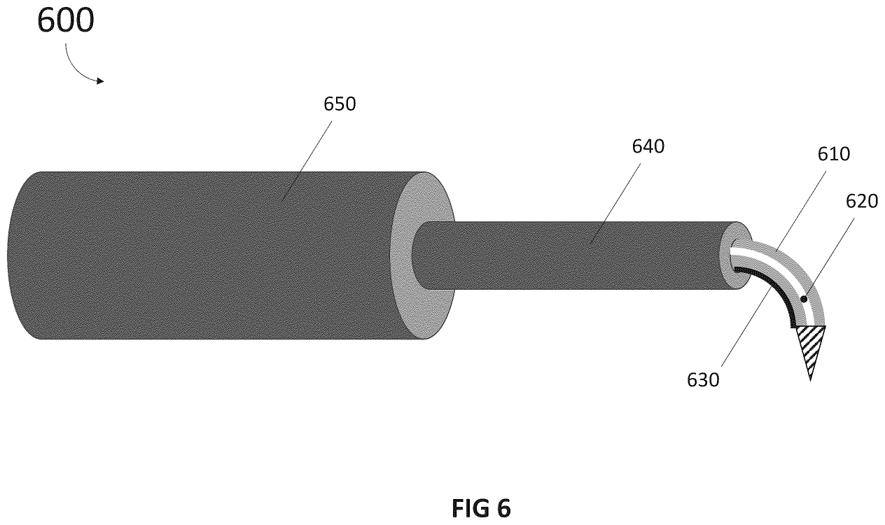

FIG. 6 is a perspective view depicting another example embodiment of tunneling device 600 for creating one or more curvilinear tunnels in a bone material. According to an aspect of the embodiments, numerous sets of drivers can control the formation of the curved tunnel to be created. According to some embodiments of tunneling device 600, a flexible hollow shaft 610 that contains a flexible drill bit 620 can be induced by a first set of one or more drivers to curve along part of or the entirety of its length by subjecting the hollow shaft to a pulling, pushing, or bending force applied lengthwise to one or more regions 630 on or near the circumference of the hollow shaft cross-section, in order to produce bending of the flexible, hollow shaft 610. This first set of one or more drivers can apply pulling, pushing, or bending force by any element either attached, affixed, or separate from the hollow shaft, or can be an integral property of the hollow shaft triggered by an external signal or stimulus. In some embodiments, the flexible hollow shaft can, based on its properties, intrinsically produce a curved configuration when extended from a rigid straight shaft 640 within which it is nested. According to some embodiments of tunneling device 600, a second set of drivers can extend and retract flexible hollow shaft 610. The head of the flexible drill bit 620 is exposed and outside the tip of the flexible hollow shaft 610. The shaft of flexible drill bit 620 rotates within flexible hollow shaft 610 and is constrained to follow any bending and curvature that is induced or generated by flexible hollow shaft 610. According to some embodiments of tunneling device 600, a third set of one or more drivers can extend or retract flexible drill bit 620, either independent of or in concert with any extension or retraction of flexible hollow shaft 610. These aspects of the example embodiment of tunneling device 600 depict no specific mechanisms for extending, retracting, and bending hollow flexible shaft 610 and flexible drill bit 620, but those with skill in the art will recognize that these numerous sets of drivers for extending and retracting the shaft and drill bit using a single or multiple pneumatic, magnetic, electrical, or mechanical actuators can be either external to or integral with device 600, and can be fully automated, partially automated, or manually powered. Device body 650 can include any mechanisms to enable the operation of device 600, and can contain any connections from external mechanisms that are necessary to operate device 600.

Referring still to FIG. 6, according to another aspect of the embodiments, tunneling is produced by the advancement of the flexible drill bit 620 either independent of, or in conjunction with the flexible hollow shaft. A motorized actuator spins flexible drill bit 620 within flexible hollow shaft 610. Although flexible hollow shaft 610 and rigid straight shaft 640 are depicted as tubes of circular cross-section with circular lumens, having mutually coincident centers, those of knowledge in the art will recognize that flexible hollow shaft 610, rigid straight shaft 640, and both their respective lumens can be of any cross-sectional shape including, but not limited to, elliptical, polygonal, or any irregular shape, and that their lumens can be positioned off-center in any location with respect to the shaft cross-section. Those of skill in the art will also recognize that hollow flexible shaft 610, flexible drill bit 620, and rigid straight shaft 640 can each be of unibody construction or can each consist of multiple parts and/or exhibit variations in material or material properties along the length or over cross-section of each part in order to allow for optimal bending properties of the flexible hollow shaft 610 and flexible drill bit 620.

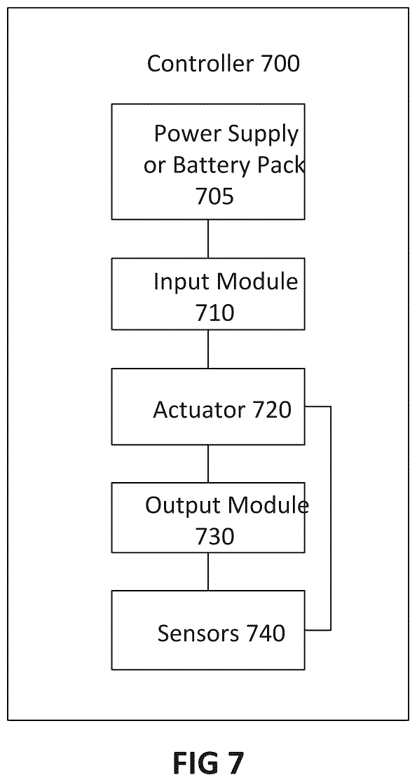

FIG. 7 shows an example block diagram for controlling the operation of an actuator in a bone tunneling device. Controller 700 comprises a power supply or battery pack 705, input module 710, actuator 720, output module 730, and sensors 740. Input module 710 enables the user to initiate the operation of actuator 720 and to adjust various parameters for the actuator's operation. These parameters can include, but are not limited to, speed and power. The actuator 720 engages the mechanism for creation of the bone tunnel. Output module 730 captures the current state of the actuator during operation. One or more sensors 740 can be used to detect changes in one or more states of the device that can require automated changes to the input module 710 in order to affect the operation of actuator 720. These sensors 740 can include, but are not limited to, temperature, position, and force.

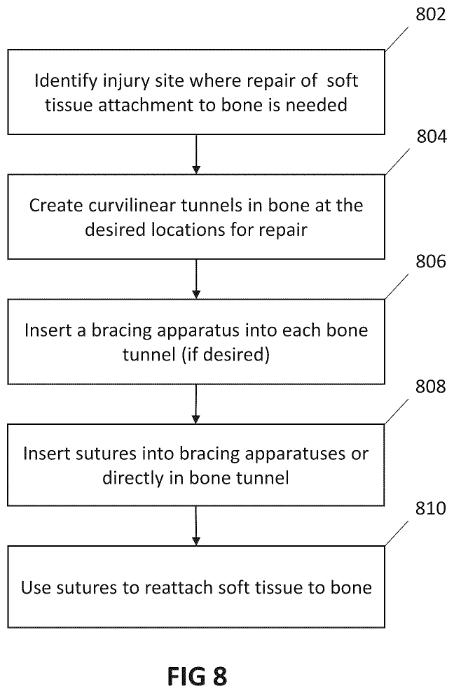

FIG. 8 illustrates an example flow chart for the method of use for the bone tunneling device in conjunction with bracing apparatuses for repairing the attachment of soft tissue to bone. After identification of the injury site (Step 802), the specific number and locations where sutures are needed to tie down the injured soft tissue are determined. A bone tunneling device is then used to create curvilinear bone tunnels at those locations in bone (Step 804). Bracing apparatuses can then be inserted into bone tunnels (Step 806). Sutures are inserted through the bracing apparatuses (Step 808) and used to tie down the soft tissue at the desired locations (Step 810). Although the insertion of bracing apparatuses is shown as a subsequent step to creating the bone tunnel, those of skill in the art will appreciate that certain embodiments of the bone tunneling device and of the bracing apparatus can enable these two steps into a single step. Likewise, although insertion of sutures through bracing apparatuses is shown as a subsequent step to insertion of the bracing apparatus into a bone tunnel, those of skill in the art will understand that certain embodiments of the tunneling device will allow for the suture to be pre-loaded into the bracing apparatus before the bracing apparatus is inserted into the bone tunnel. Those of skill in the art will also recognize that the insertion of a bracing apparatus can be omitted from the method of use, wherein sutures can be directly inserted through the bone tunnel in the absence of a bracing apparatus and subsequently used to tie down the soft tissue, or wherein the bone tunnel can be used for any other purpose.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A device for creating curvilinear tunnels in a bone, the device comprising:
a housing having a distal end configured to interface with a surface of the bone;
at least one flexible curved needle at least partially disposed within the housing,
wherein the at least one flexible curved needle is configured to be inserted into the bone and to create a curvilinear tunnel,
wherein the at least one flexible curved needle comprises a superelastic material,
wherein the at least one flexible curved needle further comprises a surface feature proximal to the curved needle tip; and
a hollow punch configured to introduce the at least one flexible curved needles at a predetermined depth under the surface of the bone;
a channel disposed within the housing, wherein the inner channel is configured to guide the at least one flexible curved needle into the bone; and
a driver configured to move the at least one flexible curved needle.

2. The device of claim 1, wherein the surface feature of the at least one flexible curved needle comprises an angled slot feature.

3. The device of claim 2, wherein the angled slot feature is disposed on a concave surface of the at least one flexible curved needle.

4. The device of claim 2, wherein the angled slot feature is disposed on a convex surface of the at least one flexible curved needle.

5. The device of claim 1, wherein the surface feature of the at least one flexible curved needle comprises a diamond-shaped orifice feature.

6. The device of claim 1, wherein the surface feature of the at least one flexible curved needle comprises an eye-shaped orifice feature.

7-39. (canceled)

40. A device for creating curvilinear tunnels in a bone, the device comprising:
a housing having a distal end configured to interface with a surface of the bone, the housing comprising one or more windows;
a plunger comprising a plunger shaft;
one or more flexible curved needles at least partially disposed within the housing, wherein the one or more flexible curved needles are configured to be inserted into the bone and to create a curvilinear tunnel, and wherein the one or more flexible curved needles comprise a superelastic material;
one or more hollow punches configured to introduce the one or more flexible curved needles at a predetermined depth under the surface of the bone;
one or more inner channels disposed within the housing, wherein each inner channel of the one or more inner channels is configured to guide a corresponding flexible curved needle of the one or more flexible curved needles into the bone; and
one or more drivers configured to move the one or more flexible curved needles,
wherein the one or more windows of the housing comprises a first window configured to provide access to an attachment point between the plunger and the one or more flexible curved needles.

41. The device of claim 40, further comprising a U-shaped insert disposed in the housing, wherein the U-shaped insert is configured to limit a travel distance of the plunger.

42. The device of claim 41, wherein the one or more windows of the housing further comprises a second window configured to receive the U-shaped insert.

43. The device of claim 41, wherein the U-shaped insert comprises two arms and a gap between the two arms.

44. The device of claim 43, wherein the plunger further comprises a neck portion, wherein a width of the neck portion is less than a width of the gap, and wherein a width of the plunger shaft is greater than the width of the gap.

45. The device of claim 40, wherein the one or more hollow punches comprise a single hollow punch, and wherein the one or more flexible curved needles comprise a single flexible curved needle.

46. The device of claim 40, wherein the one or more inner channels and the one or more hollow punches are configured to be circular in cross sectional geometry.

47. The device of claim 40, wherein the one or more inner channels and the one or more curved needles are configured to be circular in cross sectional geometry.

48. The device of claim 40, wherein the set of one or more drivers is further configured to extend the one or more hollow punches automatically.

49. The device of claim 40, wherein the set of one or more drivers is further configured to extend the one or more hollow punches by manual operation.

50. The device of claim 40, wherein the one or more hollow punches and the one or more curved needles are configured to create a single curvilinear bone tunnel.

51-61. (canceled)
